(19) United States
(12) Patent Application Publication
(10) Pub. No.: US 2007/0027519 A1
(43) Pub. Date: Feb. 1, 2007

(54) DEVICES AND METHODS FOR STENT DEPLOYMENT

(75) Inventors: Mark S. Ortiz, Milford, OH (US); Jeffrey S. Swayze, Hamilton, OH (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: ETHICON ENDO-SURGERY, INC., Cincinnati, OH (US)

(21) Appl. No.: 11/161,267

(22) Filed: Jul. 28, 2005

(51) Int. Cl. A61F 2/06

(52) U.S. Cl. 623/1.11

(57) ABSTRACT

Various methods and devices for deploying a stent a lumen are provided. In one exemplary embodiment, the stent deployment device can include an elongate shaft having at least one actuator coupled thereto and adapted to radially expand upon delivery of energy thereto. The device can also include a retractable sheath disposed therearound and adapted to maintain the stent on the actuator during deployment of the device.

### FIELD OF THE INVENTION

The present invention relates broadly to surgical devices, and in particular to methods and devices for deploying a stent.

### BACKGROUND OF THE INVENTION

Stents are generally cylindrically shaped devices that function to hold open and sometimes expand a segment of a blood vessel or other arterial lumen. Stents are usually delivered in a compressed condition to the target site and then deployed at that location into an expanded condition to support the vessel and help maintain it in an open position. They are particularly suitable for use to support and prevent compression of a bile duct that has become blocked due to cancer.

A variety of stents are known in the art. One common stent is a coiled wire stent that is expanded after being placed intraluminally. In a typical deployment procedure the stent is disposed around a balloon on a balloon catheter, and the balloon is inflated to expand stent to a larger diameter to be left in place within the body lumen at the target site. Another type of stent is a helically wound coiled spring stent that is manufactured from an expandable heat sensitive metal. Such stents manufactured from expandable heat sensitive materials allow for phase transformations of the material to occur, resulting in the expansion and contraction of the stent. A third type of stent is a self-expanding stent formed from, for example, shape memory metals or super-elastic nickel-titanium (NiTi) alloys. These stents can be delivered into a body lumen in a compressed state, and when released out of the distal end of a delivery catheter they will automatically expand from the compressed state to an expanded state.

One of the difficulties encountered in using these various stents involves delivery of the stents to a body lumen, which often has a tortuous pathway. For example, systems which rely on a “push-pull design” can experience movement of the collapsed stent within the body vessel which can lead to inaccurate positioning and, in some instances, possible perforation of the vessel wall by a protruding end of the stent. Systems which utilize an actuating wire design will tend to move to follow the radius of curvature when placed in curved anatomy of the patient. As the wire is actuated, tension in the delivery system can cause the system to straighten. As the system straightens, the position of the stent changes because the length of the catheter no longer conforms to the curvature of the anatomy. This change of the geometry of the system within the anatomy can also lead to inaccurate stent positioning.

Current stent delivery systems can also be somewhat difficult to operate with just one hand, unless a mechanical advantage system (such as a gear mechanism) is utilized. Often, deployment with one hand is desirable since it allows the physician to use his/her other hand to support a guiding catheter which is also utilized during the procedure, allowing the physician to prevent the guiding catheter from moving during deployment of the stent. Current prior art systems do not prevent axial movement of the catheters during stent deployment. Even a slight axial movement of the catheter assembly during deployment can cause inaccurate placement of the stent in the body lumen.

Accordingly, there is a need for improved methods and devices for stent deployment.

### BRIEF SUMMARY OF THE INVENTION

The present invention provides various methods and devices for deploying a stent. In one exemplary embodiment, a stent delivery device is provided that has a substantially flexible elongate shaft with a proximal end that is coupled to a handle and a distal end having an actuator disposed around a distal portion thereof. The actuator is adapted to seat a stent, and to expand upon delivery of energy thereto to deploy the stent into tissue. The device can also include a retractable sheath slidably disposed around the shaft and adapted to maintain a stent on the actuator. In use, the retractable sheath can be movable between a distal position, in which the retractable sheath is disposed around the actuator and stent, and a proximal position in which the retractable sheath is positioned proximal to the actuator and stent, thereby allowing for stent deployment.

The actuator can have a variety of configurations, and it can be formed from a variety of materials. In one exemplary embodiment, the actuator can be an electrically-expandable member, and more preferably it can be in the form of an electroactive polymer (EAP). For example, the actuator can be in the form of a fiber bundle having a flexible conductive outer shell with several electroactive polymer fibers and an ionic fluid disposed therein. Alternatively, the actuator can be in the form of a laminate having at least one flexible conductive layer, an electroactive polymer layer, and an ionic gel layer. Multiple laminate layers can be used to form a composite. The actuator can also preferably include a return electrode and a delivery electrode coupled thereto, with the delivery electrode being adapted to deliver energy to each actuator from an external energy source.

Methods for implanting a stent are also provided. In one exemplary embodiment, the method can include inserting a substantially flexible elongate shaft into a lumen and positioning an actuator having a stent disposed therearound adjacent to a target implant site. The actuator can be electrically actuated to expand radially, and thereby deploy the stent into tissue surrounding the lumen. The actuator can then be deactivated to radially contract, allowing the device to be removed from the lumen while leaving the stent in place. The device can also include a retractable sheath disposed around the actuator and stent, and the method can include retracting the sheath to expose the actuator and stent after the actuator and stent are positioned adjacent to a target implant site.

### BRIEF DESCRIPTION OF THE DRAWINGS

The invention will be more fully understood from the following detailed description taken in conjunction with the accompanying drawings, in which:

FIG. 1A is perspective view of an exemplary embodiment of a stent deployment device showing an actuator with a stent disposed therearound;

FIG. 1B is a perspective view of the distal portion of the stent deployment device shown in FIG. 1A showing a retractable sheath disposed over a stent and actuator;

FIG. 1C is a perspective view of the distal portion of the stent deployment device shown in FIG. 1B showing the stent and actuator with the retractable sheath removed;

FIG. 1D is a perspective view of the distal portion of the stent deployment device shown in FIG. 1C showing the actuator expanded to deliver the stent;

FIG. 2A is a cross-sectional view of a prior art fiber bundle type EAP actuator;

FIG. 2B is a radial cross-sectional view of the prior art actuator shown in FIG. 2A;

FIG. 3A is a cross-sectional view of a prior art laminate type EAP actuator having multiple EAP composite layers;

FIG. 3B is a perspective view of one of the composite layers of the prior art actuator shown in FIG. 3A;

FIG. 4A is an illustrate showing the stent deployment device of FIG. 1A in use, showing the actuator disposed within a lumen and having a stent and the retractable sheath disposed therearound;

FIG. 4B is an illustration showing the stent deployment device of FIG. 4A, showing the sheath retracted to expose the stent and actuator; and

FIG. 4C is an illustration showing the stent deployment device of FIG. 4B, showing the actuator expanded to deliver the stent into the tissue.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides methods and devices for deploying a stent in bodily lumens, such as the carotid arteries, peripheral vessels, urethra, esophagus, bile duct, jejunum, and duodenum. In an exemplary embodiment, the stent deployment device can include one or more actuators for seating a stent(s) therearound. The actuator(s) can be adapted to radially expand to effect deployment of the stent(s). A person skilled in the art will appreciate that the methods and devices disclosed herein can have a variety of configurations, and they can be adapted for use in a variety of medical procedures. Moreover, the methods and devices disclosed herein can be used with any other procedures known in the art that require the placement of stents. The device can also be incorporated into a variety of other devices to allow stent delivery to be performed in conjunction with other procedures.

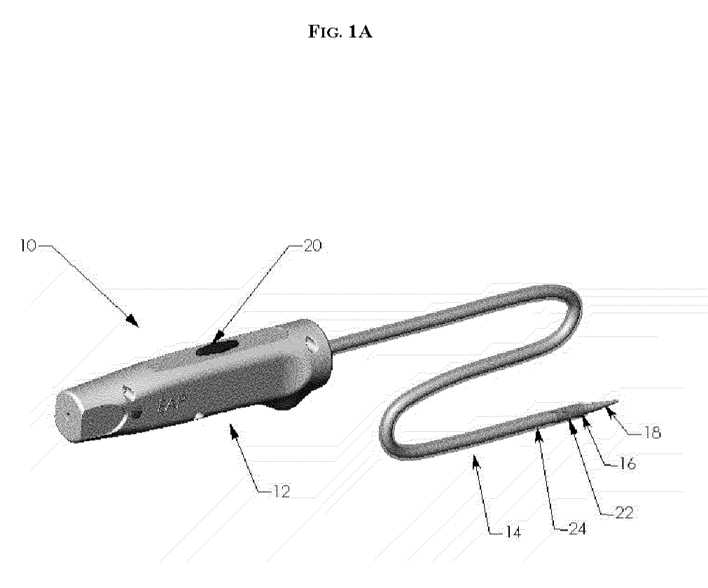

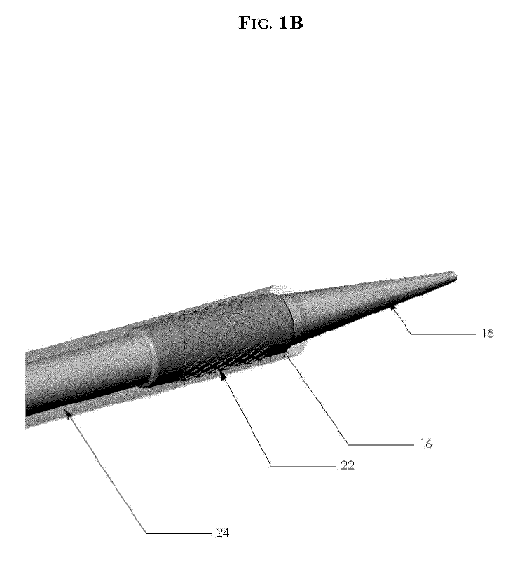

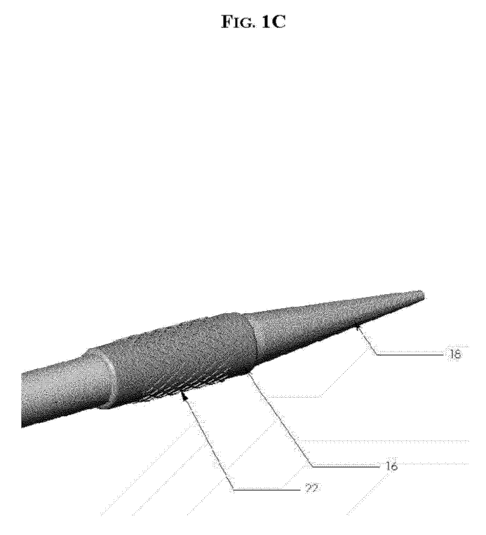

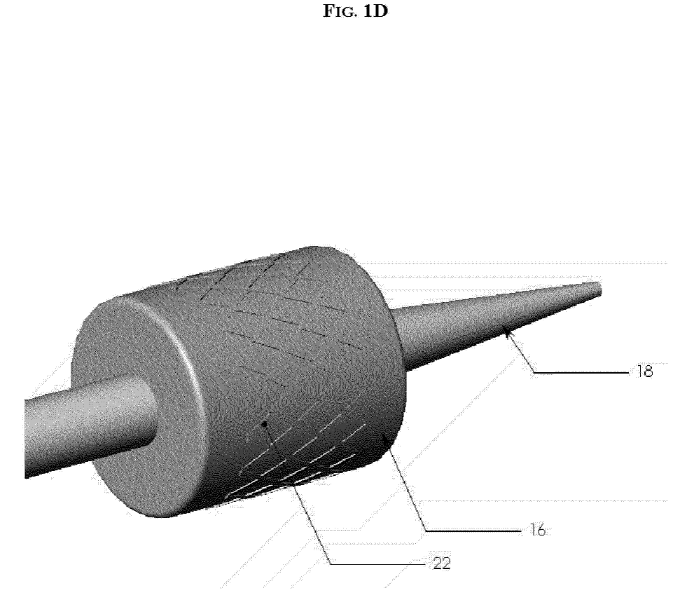

FIGS. 1A-1D illustrate one exemplary embodiment of a stent deployment device 10 that is adapted to deploy a stent 22 into a lumen. The device 10 can have a variety of configurations, but in one exemplary embodiment the device 10 can include a handle 12, an elongate shaft 14 having a proximal end 14a coupled to the handle 12 and a distal end 14b adapted to be positioned within a lumen, and an actuator 16 disposed around a distal portion of the elongate shaft 14. A retractable sheath 24 can optionally be slidably disposed around the actuator 16 and stent 22 to maintain the stent 22 on the actuator 16 in a fixed position during insertion of the elongate shaft 14 through the lumen.

The handle 12 can have any configuration that allows a user to manually control the device 10, and in particular to control energy delivery and retraction of the sheath 24, as will be discussed in more detail below. As shown in FIG. 1A, the handle 12 has a generally elongate shape to facilitate grasping. The handle 12 can also include features and components to facilitate operation of the device 10. For example, in one exemplary embodiment, an energy source, such as a battery, can be disposed within the handle 12 for delivering energy to the actuator 16. Alternatively, the handle 12 can be adapted to be coupled to an energy source, such as an electrical outlet. The handle 16 can also include a mechanism that allows a user selectively activate and deactivate the delivery of energy to the actuator 16. For example, the handle 12 can include a button 20 that can be moved or pressed to deliver energy to the actuator 16, as shown in FIG. 1A. Alternatively, or in addition, the handle 12 can include a sliding lever or rotating dial that can be used to control the amount of energy being delivered, thereby allowing the amount of expansion of the actuator 16 to be controlled, as will be discussed in more detail below.

The elongate shaft 14 extending from the handle 12 can also have a variety of configurations, and the shape and the size of the elongate shaft 14 can vary depending upon the intended use of the device 10. In one exemplary embodiment, the elongate shaft 14 can have a generally cylindrical shape and it can be flexible to allow for insertion into the esophagus. The length of the shaft 14 can vary depending upon the particular procedure being performed. For example, where a stent is deployed in a bile duct, the shaft 14 can have a length in the range of about 4 feet to 6 feet. The elongate shaft 14 can also include various features to facilitate insertion through a lumen, such as a tapered distal tip 18. A person skilled in the art will appreciate that the shaft can be rigid, and it can have a variety of other configurations. For example, while not shown, the shaft 14 can include a lumen extending therethrough for providing access to a surgical site, such as for drug delivery, imaging, fluid flow, etc.

As previously indicated, the device 10 can also include one or more actuators coupled to the flexible elongate shaft 14 to effect stent deployment. While the actuator(s) can have a variety of configurations, one suitable actuator is an electroactive polymer actuator. Electroactive polymers (EAPs), also referred to as artificial muscles, are materials that exhibit piezoelectric, pyroelectric, or electrostrictive properties in response to electrical or mechanical fields. In particular, EAPs are a set of conductive doped polymers that change shape when an electrical voltage is applied. The conductive polymer can be paired with some form of ionic fluid or gel using electrodes. Upon application of a voltage potential to the electrodes, a flow of ions from the fluid/gel into or out of the conductive polymer can induce a shape change of the polymer. Typically, a voltage potential in the range of about 1V to 4 kV can be applied depending on the particular polymer and ionic fluid or gel used. It is important to note that EAPs do not change volume when energized, rather they merely expand in one direction and contract in a transverse direction.

One of the main advantages of EAPs is the possibility to electrically control and fine-tune their behavior and properties. EAPs can be deformed repetitively by applying external voltage across the EAP, and they can quickly recover their original configuration upon reversing the polarity of the applied voltage. Specific polymers can be selected to create different kinds of moving structures, including expanding, linear moving, and bending structures. The EAPs can also be paired to mechanical mechanisms, such as springs or flexible plates, to change the effect of the EAP on the mechanical mechanism when voltage is applied to the EAP. The amount of voltage delivered to the EAP can also correspond to the amount of movement or change in dimension that occurs, and thus energy delivery can be controlled to effect a desired amount of change.

There are two basic types of EAPs and multiple configurations for each type. The first type is a fiber bundle that can consist of numerous fibers bundled together to work in cooperation. The fibers typically have a size of about 30-50 microns. These fibers may be woven into the bundle much like textiles and they are often referred to as EAP yarn. In use, the mechanical configuration of the EAP determines the EAP actuator and its capabilities for motion. For example, the EAP may be formed into long strands and wrapped around a single central electrode. A flexible exterior outer sheath will form the other electrode for the actuator as well as contain the ionic fluid necessary for the function of the device. When voltage is applied thereto, the EAP will swell causing the strands to contract or shorten. An example of a commercially available fiber EAP material is manufactured by Santa Fe Science and Technology and sold as PANION™ fiber and described in U.S. Pat. No. 6,667,825, which is hereby incorporated by reference in its entirety.

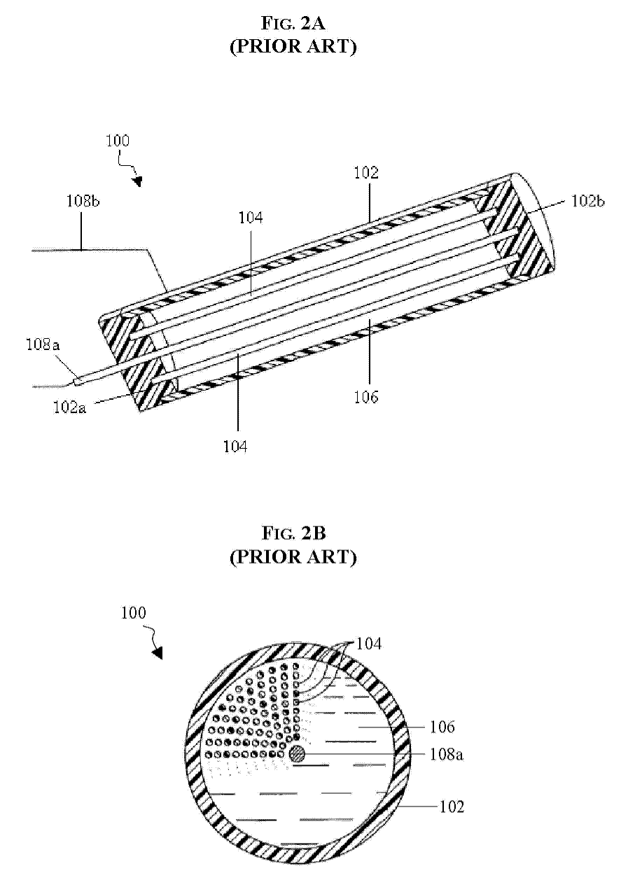

FIGS. 2A and 2B illustrate one exemplary embodiment of an EAP actuator 100 formed from a fiber bundle. As shown, the actuator 100 generally includes a flexible conductive outer sheath 102 that is in the form of an elongate cylindrical member having opposed insulative end caps 102a, 102b formed thereon. The conductive outer sheath 102 can, however, have a variety of other shapes and sizes depending on the intended use. As is further shown, the conductive outer sheath 102 is coupled to a return electrode 108a, and an energy delivering electrode 108b extends through one of the insulative end caps, e.g., end cap 102a, through the inner lumen of the conductive outer sheath 102, and into the opposed insulative end cap, e.g., end cap 102b. The energy delivering electrode 108b can be, for example, a platinum cathode wire. The conductive outer sheath 102 can also include an ionic fluid or gel 106 disposed therein for transferring energy from the energy delivering electrode 108b to the EAP fibers 104, which are disposed within the outer sheath 102. In particular, several EAP fibers 104 are arranged in parallel and extend between and into each end cap 102a, 120b. As noted above, the fibers 104 can be arranged in various orientations to provide a desired outcome, e.g., radial expansion or contraction, or bending movement. In use, energy can be delivered to the actuator 100 through the active energy delivery electrode 108b and the conductive outer sheath 102 (anode). The energy will cause the ions in the ionic fluid to enter into the EAP fibers 104, thereby causing the fibers 104 to expand in one direction, e.g., radially such that an outer diameter of each fiber 104 increases, and to contract in a transverse direction, e.g., axially such that a length of the fibers decreases. As a result, the end caps 102a, 120b will be pulled toward one another, thereby contracting and decreasing the length of the flexible outer sheath 102.

Another type of EAP is a laminate structure, which consists of one or more layers of an EAP, a layer of ionic gel or fluid disposed between each layer of EAP, and one or more flexible conductive plates attached to the structure, such as a positive plate electrode and a negative plate electrode. When a voltage is applied, the laminate structure expands in one direction and contracts in a transverse or perpendicular direction, thereby causing the flexible plate(s) coupled thereto to shorten or lengthen, or to bend or flex, depending on the configuration of the EAP relative to the flexible plate(s). An example of a commercially available laminate EAP material is manufactured by Artificial Muscle Inc, a division of SRI Laboratories. Plate EAP material, referred to as thin film EAP, is also available from EAMEX of Japan.

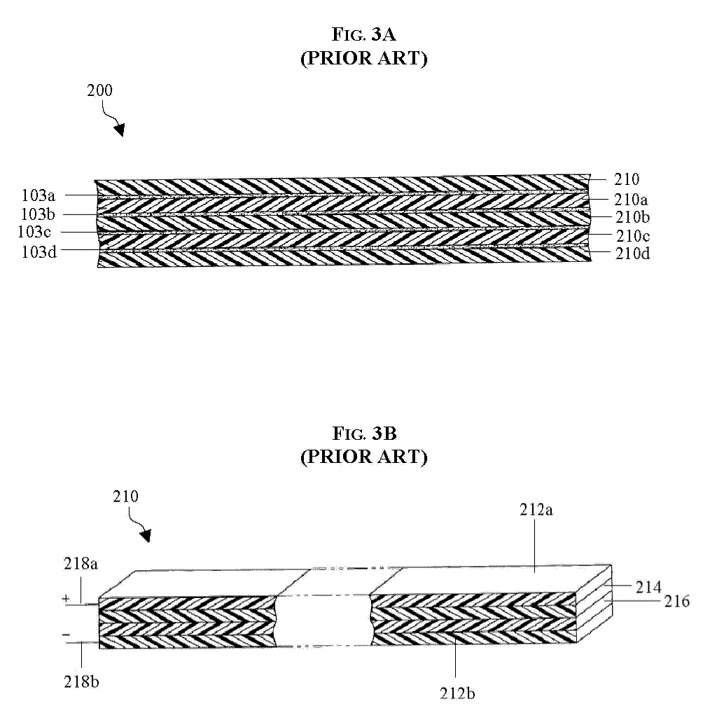

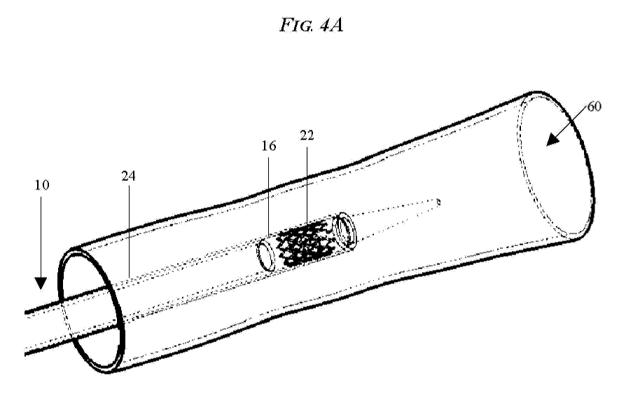

FIGS. 3A and 3B illustrate an exemplary configuration of an EAP actuator 200 formed from a laminate. Referring first to FIG. 3A, the actuator 200 can include multiple layers, e.g., five layers 210, 210a, 210b, 210c, 210d are shown, of a laminate EAP composite that are affixed to one another by adhesive layers 103a, 103b, 103c, 103d disposed therebetween. One of the layers, i.e., layer 210, is shown in more detail in FIG. 3B, and as shown the layer 210 includes a first flexible conductive plate 212a, an EAP layer 214, an ionic gel layer 216, and a second flexible conductive plate 212b, all of which are attached to one another to form a laminate composite. The composite can also include an energy delivering electrode 218a and a return electrode 218b coupled to the flexible conductive plates 212a, 212b, as further shown in FIG. 3B. In use, energy can be delivered to the actuator 200 through the active energy delivering electrode 218a. The energy will cause the ions in the ionic gel layer 216 to enter into the EAP layer 214, thereby causing the layer 214 to expand in one direction and to contract in a transverse direction. As a result, the flexible plates 212a, 212b will be forced to flex or bend, or to otherwise change shape with the EAP layer 214.

Referring back to FIGS. 1A-1D, either type of actuator can be adapted to seat a stent to effect deployment thereof. However, in an exemplary embodiment, the actuator 16 is in the form of an EAP laminate, or a composite formed from multiple laminates. While the number and location of the actuator 16 can vary, in the illustrated embodiment the elongate shaft 14 includes a single actuator 16 coupled to a distal end portion of the shaft 16 just proximal to the tapered tip 18. The actuator 16 an be mated to the shaft 14 using a variety of techniques, and the mating technique can vary depending on the type of actuator. Where the actuator 16 is an EAP laminate or composite actuator, the actuator 16 can be wrapped around and adhered to the shaft 14 using an adhesive or other mating technique. The orientation of the EAP actuator can be configured to allow the actuator 16 to expand radially and contract axially when energy is delivered thereto, thereby allowing a diameter of the actuator 16 to increase. While not shown, the actuator 16 can optionally be disposed within an inner lumen of the shaft and/or embedded within the walls of the shaft 14.

In use, energy can be delivered to the actuator 16 to cause the actuator to expand radially and contract axially. While various techniques can be used to deliver energy to the actuator 16, in one embodiment the actuator can be coupled to a return electrode and a delivery electrode that is adapted to communicate energy from an external power source to the actuator. The electrodes can extend through the inner lumen in the elongate shaft 14, be embedded in the sidewalls of the elongate shaft 14, or they can extend along an external surface of the elongate shaft 14.

As previously indicated, the device 10 can also optionally include a retractable sheath 24 that is slidably disposed around the actuator 16 and the stent 22 to maintain the placement of the stent 22 during insertion into a lumen. The sheath 24 can have a variety of configurations, but in one embodiment the sheath 24 can have a generally elongate hollow tubular shape that is sized to fit around the shaft and the actuator 16 in the unexpanded state. The sheath 24 can be slidably coupled to the device 10 using virtually any technique known in the art. By way of non-limiting example, the sheath 24 can be coupled to a lever disposed within the handle 12 such that movement of the lever moves the sheath. Alternatively, the sheath can be retracted using a wire, or an EAP that contracts axially to pull the sheath proximally. In use, the sheath 24 is adapted to move between a distal position in which the sheath 24 is disposed around the actuator 16 and the stent 22, and a proximal position in which the sheath 24 is positioned proximal to the actuator 16 and stent 22 to allow stent 22 deployment.

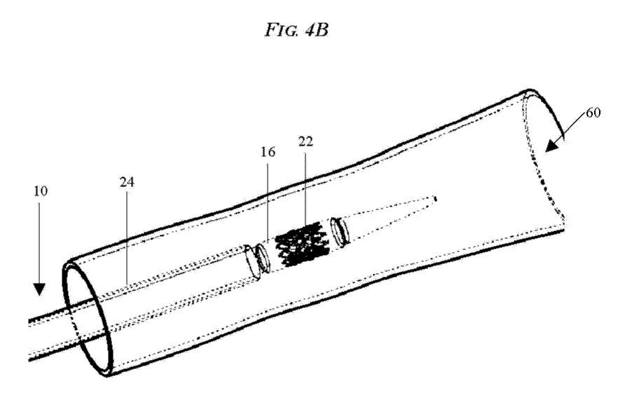

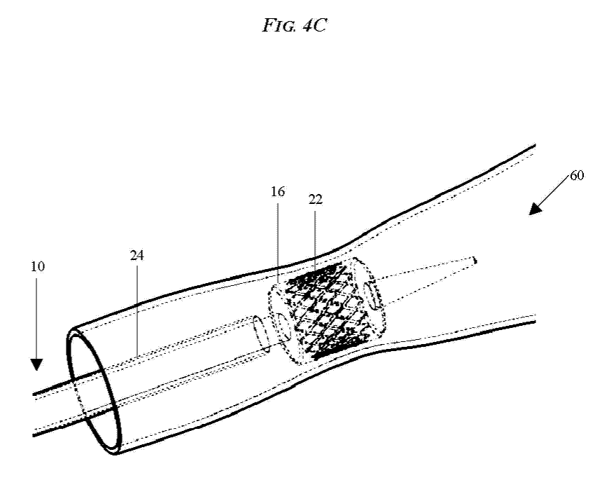

FIGS. 4A-4C illustrate exemplary methods for using a deployment device to deliver a stent. A person skilled in the art will appreciate that any type of stent can be used, but preferably the stent is of the type that is adapted to expand in conjunction with the radial expansion of the actuator 16, and to remain in the expanded state, thereby allowing the stent to remain in engagement with the lumen wall. In one exemplary embodiment, the stent can be formed from a wire, such as a wire mesh stents, woven wire stents, and wire cut stents.

As shown in FIG. 4A, the device 10 is inserted into a lumen 60 in the body in a normal longitudinal or linear configuration with the actuator 16 being deactivated, i.e., in a resting configuration without energy being applied thereto, and with a stent 22 placed around the actuator 16. The retractable sheath 24 can extend over actuator 16 and the stent 22 in order to facilitate insertion through the lumen. Once the target site is located, for example, by imaging the lumen, the actuator is positioned at the desired implant site, and the retractable sheath 24, if used, is retracted to expose the actuator 16 and the stent 22, as shown in FIG. 4B. Energy can then be delivered to the actuator 16 to cause the actuator 16 to radially expand, as shown in FIG. 4C. The amount of radial expansion of the actuator 16 can be controlled by adjusting the amount of energy being delivered, and the radial expansion of the actuator 16 can be maintained so long as the energy is continuously supplied to the actuator 16. As a result of the radial expansion of the actuator 16, the stent 22 will also radially expand such that it engages the inner surface of a lumen. Typically the actuator 16 can expand at least about 30% its size when energy is delivered thereto. For example, in certain exemplary embodiments the actuator 16 can have a diameter that ranges from about 16 mm in the unexpanded condition to about 25 mm in the expanded condition. The shape and size of the actuator 16 can, of course, vary depending on the intended use. Once the stent 22 is delivered, energy delivery to the actuator 16 can be terminated to cause the actuator 16 to return to its resting configuration, and to allow for removal of the device 10 from the lumen. If the device includes more than one actuator formed thereon, other actuators can also be selectively activated and de-activated, either alone or in combination, to effect stent deployment. Following stent delivery and de-actuation of the actuator, the sheath can be placed around the actuator to facilitate removal from the lumen.

One skilled in the art will further appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A stent delivery device, comprising:
a substantially flexible elongate shaft having a proximal end coupled to a handle and a distal end having an actuator disposed around a distal portion thereof and adapted to radially expand upon delivery of energy thereto; and
a retractable sheath slidably disposed around the actuator.

2. The device of claim 1, wherein the actuator is adapted to receive a stent disposed therearound, and the retractable sheath is adapted to engage the actuator to maintain the stent in a fixed position.

3. The device of claim 1, wherein the retractable sheath is movable between a distal position, in which the retractable sheath is disposed around the actuator, and a proximal position in which the retractable sheath is positioned proximal to the actuator.

4. The device of claim 1, wherein the actuator comprises an electroactive polymer.

5. The device of claim 1, wherein the actuator comprises a flexible conductive outer shell having an electroactive polymer and an ionic fluid disposed therein.

6. The device of claim 1, wherein the actuator comprises at least one electroactive polymer composite having at least one flexible conductive layer, an electroactive polymer layer, and an ionic gel layer.

7. The device of claim 1, wherein the actuator includes a return electrode and a delivery electrode coupled thereto, the delivery electrode being adapted to deliver energy to the actuator from an energy source.

8. The device of claim 7, further comprising an energy source disposed within the handle and coupled to the delivery electrode.

9. A delivery device and implant system, comprising:
a handle;
a flexible elongate shaft extending from the handle;
an electrically-expandable member disposed around a distal portion of the flexible elongate shaft and configured to radially expand when energy is delivered thereto;
a stent disposed around the electrically-expandable member; and
a retractable sheath slidably disposed around the electrically-expandable member and the stent.

10. The system of claim 9, wherein the stent is formed from a wire.

11. The system of claim 9, wherein the retractable sheath is adapted to engage the electrically-expandable member to maintain the stent in a fixed position.

12. The system of claim 9, wherein the retractable sheath is movable between a distal position, in which the retractable sheath is disposed around the electrically-expandable member, and a proximal position, in which the retractable sheath is positioned proximal to the electrically-expandable member.

13. The system of claim 9, wherein the electrically-expandable member comprises an electroactive polymer.

14. The system of claim 9, wherein the electrically-expandable member comprises a flexible conductive outer shell having an electroactive polymer and an ionic fluid disposed therein.

15. The system of claim 9, wherein electrically-expandable member comprises at least one electroactive polymer composite having at least one flexible conductive layer, an electroactive polymer layer, and an ionic gel layer.

16. The system of claim 9, wherein the electrically-expandable member includes a return electrode and a delivery electrode coupled thereto, the delivery electrode being adapted to deliver energy to the electrically-expandable member from an energy source.

17. The system of claim 16, further comprising an energy source disposed within the handle and coupled to the delivery electrode.

18. A method for implanting a stent, comprising:
inserting a substantially flexible elongate shaft into a lumen;
positioning an actuator located on a distal portion of the flexible elongate shaft within a lumen, the actuator having a stent disposed therearound; and
electrically actuating the actuator to cause the actuator to expand radially, thereby deploying the stent into tissue surrounding the lumen.

19. The method of claim 18, further comprising retracting a sheath surrounding the actuator and the stent such that the actuator and the stent are exposed within the lumen.

20. The method of claim 18, wherein electrically actuating the actuator causes a diameter of the stent to increase such that the stent engages an inner wall of the lumen.

21. The method of claim 18, further comprising electrically de-actuating the actuator to cause the actuator to radially contract.

22. The method of claim 18, further comprising inserting the substantially flexible elongate shaft into an esophagus.
